(19) United States
(12) Patent Application Publication
(10) Pub. No.: US 2019/0204321 A1
(43) Pub. Date: Jul. 4, 2019

(54) FIELD EFFECT SENSOR FOR COLON CANCER

(71) Applicants: KOREA INSTITUTE OF SCIENCE AND TECHNOLOGY, Seoul (KR); THE ASAN FOUNDATION, Seoul (KR); UNIVERSITY OF ULSAN FOUNDATION FOR INDUSTRY COOPERATION, Ulsan (KR)

(72) Inventors: Kwan Hyi LEE, Seoul (KR); Min Hong JEUN, Seoul (KR); Sung Wook PARK, Seoul (KR); Seung Jae MYUNG, Seoul (KR); Choung Soo KIM, Seoul (KR); Sang Yeob KIM, Seoul (KR); Eun Ju DO, Seoul (KR); Ja Young KANG, Seoul (KR)

(73) Assignees: KOREA INSTITUTE OF SCIENCE AND TECHNOLOGY, Seoul (KR); THE ASAN FOUNDATION, Seoul (KR); UNIVERSITY OF ULSAN FOUNDATION FOR INDUSTRY COOPERATION, Ulsan (KR)

(21) Appl. No.: 16/314,253

(22) PCT Filed: Jul. 20, 2017
(86) PCT No.: PCT/KR2017/007835

§ 371 Date: Mar. 18, 2019

(30) Foreign Application Priority Data
Jul. 20, 2016 (KR) 10-2016-0091997

(51) Int. Cl. G01N 33/574; G01N 33/543; G01N 27/414

(52) U.S. Cl. CPC G01N 33/57419; G01N 33/54386; G01N 27/4145; G01N 2800/50; G01N 27/4146; G01N 33/57484; G01N 33/5438

(57) ABSTRACT

The present invention relates to a high-sensitivity liquid field-effect sensor for colon cancer, applicable to a sample such as blood or stool. The sensor according to one aspect enables ultra-high precision/low-concentration detection of colon cancer biomarkers, thereby having an effect of enabling early diagnosis of colon cancer even with a very small amount of a sample.

### TECHNICAL FIELD

The present disclosure relates to a high-sensitivity liquid field-effect sensor for colorectal cancer, applicable to a sample such as blood or stool, a kit for diagnosing colorectal cancer including the sensor, and a method of diagnosing colorectal cancer.

### BACKGROUND ART

Colorectal cancer is the third most common cancer in men (746,000 cases in the year 2012) and the second most common cancer in women (614,000 cases in the year 2012). Colorectal cancer causes 694,000 deaths in 2012, accounting for 8.5% of cancer mortality overall. Colorectal cancer is a cancer that frequently occurs in the developed world, including the United States, Europe, etc. In Korea, the number of colorectal cancer patients is rapidly increasing in accordance with dietary changes. The incidence of colorectal cancer among Korean men ranked first among all Asian countries and fourth worldwide, and has currently reached an extremely dangerous level. It is estimated that the incidence rate of colorectal cancer will nearly double by 2030. When found early, colorectal cancer is highly curable (90% or more). However, there are no symptoms at the early stage, and it is much more likely to be diagnosed at a later advanced stage, unlike other cancers. 51.6% of colorectal cancer patients are diagnosed at stage 3 or 4, and therefore, there is a need for early diagnosis of colorectal cancer.

Meanwhile, the technological trends in the current in-vitro diagnostics market are rapidly shifting to molecular diagnostic technologies. Important technical factors in molecular diagnostics may be categorized into discovery of nucleic acid biomarkers for molecular diagnostics, which are indicators of a particular disease, and a biomarker detection technology capable of detecting biomarkers with high sensitivity and specificity. Discovery of molecular diagnostic biomarkers which may be used to diagnose a particular disease with high sensitivity and specificity is an underlying technology that may be applied as it is to various detection systems, and is a knowledge-intensive technology that may be commercialized in a short time.

The US FDA has approved several tumor-associated antigens for cancer diagnostic purposes, including prostate-cancer antigen (PSA) for diagnosis of prostate cancer, carcinoembryonic antigen (CEA) for diagnosis of colorectal cancer, alpha-fetoprotein (AFP) for diagnosis of testicular cancer and liver cancer, etc. Molecular diagnostic methods used to determine a therapeutic direction using a patient's cancer tissue includes MammaPrint, OncotypeDx, etc. approved by the US FDA, or products commercialized at CLIA level. However, there are very few cases of commercialized molecular diagnostic technologies using major body fluid samples such as blood/urine/sputum, etc.

Accordingly, there is a need for a technique capable of detecting a substance expressed in the early stage of colorectal cancer cells in a body fluid sample such as blood, etc. with specificity and high sensitivity.

### DESCRIPTION OF EMBODIMENTS

### Technical Problem

Accordingly, the present disclosure provides a high-precision field-effect diagnostic sensor for colorectal cancer that enables early or timely diagnosis of colorectal cancer by detecting colorectal cancer biomarkers in a sample such as blood, stool, etc.

Further, in the present disclosure, blood or stool which may be relatively easily obtained may be used in the diagnosis, thereby minimizing a patient's discomfort in and adverse effects of existing diagnostic tests for colorectal cancer such as colonoscopy and providing a quick and simple patient-friendly technology for monitoring/diagnosing a disease, and thus the present disclosure may replace existing invasive diagnostic techniques.

In particular, colorectal cancer secreted protein (CCSP) used as a biomarker of the present disclosure is a serum marker detectable at the early stage of colorectal cancer and exhibits a mean of a 78-fold increase in expression, as compared with that of a normal colon. At present, there are few biomarkers suitable for the adenoma stage of colorectal cancer, but CCSP shows high expression levels in adenoma. The present disclosure enables ultra-high precision/low-concentration detection, thereby realizing early diagnosis of colorectal cancer.

Further, CCSP is not detectable by known ELISA methods, whereas the sensor of the present disclosure may detect CCSP, because the sensor enables ultra-high precision/low-concentration detection.

### Solution to Problem

An aspect provides a diagnostic sensor for colorectal cancer, the sensor including a sensing unit for detecting an analyte in a sample, and a signal processing unit that is electrically connected to the sensing unit, the signal processing unit including an ion-sensitive field-effect transistor.

In one specific embodiment, the sensor may include an electrochemical sensing unit for detecting an analyte in a sample, and a signal processing unit for amplifying signals generated from the sensing unit, the signal processing unit being electrically connected to the sensing unit and including an ion-sensitive field-effect transistor, wherein the sensing unit may be separable from the signal processing unit, and the connection may be made between an electrode of the sensing unit and an upper gate electrode of the transistor.

In another specific embodiment, the sensor may further include a connecting portion for connecting the sensing unit and the signal processing unit. The connecting portion may be configured such that the sensing unit is separable from the connecting portion, for example, the connecting portion may have a form of a plug.

In still another specific embodiment, the sensor may further include a display unit for displaying results. The display unit may further include a display for displaying results and a frame including one or more control interfaces (e.g., a power button, a scroll wheel, etc.). The frame may include a slot for receiving the sensor. The frame may include a circuit thereinside to apply a potential or current to an electrode of the sensor when a sample is provided. A suitable circuit for the meter may be, for example, a suitable voltage meter capable of measuring a potential crossing the electrode. Also, provided is a switch that is opened when the potential is measured or is closed when the current is measured. The switch may be a mechanical switch (e.g., relay) or a solid-state switch. The circuit may be used to measure a potential difference or a current difference. As understandable to one of ordinary skill in the art, other circuits including more simple or complicated circuits may be used to apply a potential difference, a current, or both of them.

The sensing unit may include a substrate; a working electrode and a reference electrode formed on the substrate; an analyte-binding material immobilized on the working electrode; and a test cell for accommodating the electrode, the analyte-binding material, and the analyte. The sensing unit may be configured to be disposable. For example, the substrate may be a substance selected from the group consisting of a silicone, a glass, a metal, a plastic, and a ceramic. Specifically, the substrate may be selected from the group consisting of a silicone, a glass, a polystyrene, a polymethyl acrylate, a polycarbonate, and a ceramic. Examples of the electrode may include titanium nitride, silver, silver epoxy, palladium, copper, gold, platinum, silver/silver chloride, silver/silver ion, or mercury/mercury oxide. Further, the sensing unit may include an insulating electrode formed on the substrate or the working electrode. The insulating electrode may include a naturally or artificially formed oxide film. Examples of the oxide film may include Si₃O_{y}, HₓfO_{y}, AlₓO_{y}, Taₓo_{y}, or TiₓO_{y} (wherein x or y is an integer of 1 to 5). Formation of the oxide film may be performed by a known method. For example, an oxide may be deposited on a substrate by liquid phase deposition, evaporation, or sputtering.

As used herein, the term “analyte-binding material” or “analyte-binding reagent” may be used interchangeably, and may refer to a material capable of providing the sensing unit with functionalization or a material capable of specifically binding to an analyte. The analyte-binding material may include DNAs, RNAs, nucleotides, nucleosides, proteins, polypeptides, peptides, amino acids, carbohydrates, enzymes, antibodies, antigens, receptors, viruses, substrates, ligands, membranes, or a combination thereof. For example, the analyte-binding material may be an antibody capable of specifically binding to colorectal cancer secreted protein (CCSP) such as CCSP-2, or carcinoembryonic antigen (CEA), each of which is a diagnostic marker for colorectal cancer. Therefore, the diagnostic sensor for colorectal cancer may be a sensor for detecting a diagnostic biomarker for colorectal cancer, for example, CCSP or CEA. Further, the analyte-binding material may include a redox enzyme. The redox enzyme may refer to an enzyme oxidizing or reducing a substrate, and example thereof may include oxidase, peroxidase, reductase, catalase, and dehydrogenase. Example of the redox enzyme may include glucose oxidase, lactate oxidase, cholesterol oxidase, glutamate oxidase, horseradish peroxidase (HRP), alcohol oxidase, glucose oxidase (GOx), glucose dehydrogenase (GDH), cholesterol esterase, ascorbic acid oxidase, alcohol dehydrogenase, laccase, tyrosinase, galactose oxidase, and bilirubin oxidase. The analyte-binding material may be immobilized on the substrate, working electrode, or insulating electrode, and the term “immobilized” may refer to a chemical or physical binding between the analyte-binding material and the substrate. Further, an immobilization compound may be immobilized on the substrate or electrode. The immobilization compound may refer to a material capable of binding with the analyte or a linker for immobilizing the analyte-binding material on the surface of the substrate or electrode. The immobilization compound may include biotin, avidin, streptavidin, a carbohydrate, poly L-lysine, a compound having a hydroxyl group, a thiol group, an amine group, an alcohol group, a carboxyl group, an amino group, a sulfur group, an aldehyde group, a carbonyl group, a succinimide group, a maleimide group, an epoxy group, or an isothiocyanate group, or a combination thereof.

As used herein, the term “analyte” may refer to a material of interest which may be present in a sample. The detectable analyte may include materials involved in a specific binding interaction with one or more analyte-binding materials, which participate in a sandwich, competitive, or replacement assay configuration. Examples of the analyte may include antigens such as peptides (e.g., hormones) or haptens, proteins (e.g., enzymes), carbohydrates, proteins, drugs, agricultural chemicals, microorganisms, antibodies, and nucleic acids participating in sequence-specific hybridization with complementary sequences. More specific examples of the analyte may include CCSP such as CCSP-2, or CEA, each of which is a diagnostic marker for colorectal cancer.

The sample may be a biological sample derived from a subject, for example, a mammal including a human. Further, the biological sample may be blood, whole blood, serum, plasma, lymphatic fluid, urine, stool, a tissue, a cell, an organ, a bone marrow, saliva, sputum, cerebrospinal fluid, or a combination thereof.

Further, the colorectal cancer may include lymphoma, sarcoma, or squamous cell carcinoma, in addition to adenoma that occurs in the colorectal mucosa.

In the sensing unit, the sample may enter through the test cell for accommodating the electrode, the analyte-binding material, and the analyte, and an analyte present in the sample may bind with the analyte-binding material to cause a chemical potential gradient in the test cell. The term “chemical potential gradient” may mean a concentration gradient of an active species. When the gradient is present between two electrodes, a potential difference may be detectable when a circuit is opened, and when the circuit is closed, a current may flow until the gradient is reduced to zero. The chemical potential gradient may be a potential difference between the electrodes or a potential gradient occurring due to the providing of a current flow. The test cell may be prepared from polydimethylsiloxane (PDMS), polyethersulfone (PES), poly(3,4-ethylenedioxythiophene), poly(styrenesulfonate), polyimide, polyurethane, polyester, perfluoropolyether (PFPE), polycarbonate, or a combination of the polymers.

The ion-sensitive field-effect transistor may include a lower gate electrode; a lower insulating layer provided on the lower gate electrode; a source and a drain, provided on the lower insulating layer and separated from each other; a channel layer provided on the lower insulating layer and arranged between the source and the drain; an upper insulating layer formed on the source, the drain, and the channel layer; and an upper gate electrode formed on the upper insulating layer.

A small surface potential voltage difference that occurs in the sensing unit significantly amplifies a threshold voltage variation of a lower field effect transistor due to super electrostatic coupling generated in a dual gate ion-sensitive field-effect transistor (ISFET) including a channel layer. In this regard, an amplification factor may be determined according to a thickness of the lower insulating layer, a thickness of the channel layer, and a thickness of the insulating layer of the upper gate. As the thickness of the lower insulating layer increases, and the thickness of the upper insulating layer and the thickness of the channel layer decrease, the amplification factor may become larger.

The channel layer may be an ultra-thin film layer having a thickness, for example, of 10 nm or less, 9 nm or less, 8 nm or less, 7 nm or less, 6 nm or less, 5 nm or less, or 4 nm or less.

When the thickness of the channel layer is within any of these ranges, super electrostatic coupling, which may control under all conditions up to the upper interface, may occur due to a strong electric field of the lower gate electrode induced at the ultra-thin film. As a result, electrons and holes induced at an upper gate interface may be controlled, and current leakage may be blocked. In addition, by permitting a stable amplification factor, a linear response, hysteresis, and a drift phenomenon depending on a surface potential may be improved, and the electrostatic coupling of the upper and lower gates may be sustained. Further, when the thickness of the channel layer is within any of the above ranges, a transistor including the ultra-thin channel layer may, as compared with an existing transistor, have increased ion-sensing ability while permitting a larger amplification factor. Further, when the thickness of the channel layer is within any of the above ranges, a transistor including the ultra-thin channel layer may, as compared with an existing transistor, have improved stability. The varying amplification factor seen in a thick channel layer may cause deterioration of a device due to ion damage, by combination with the current leakage induced at an upper interface. On the other hand, a transistor according to a specific embodiment, in which the current leakage is controlled while permitting a constant amplification factor, may minimize an effect of ion damage. In addition, when the lower insulating layer is excessively thick in an existing transistor, a lower electric field may not fully control a channel region, and thus the electrostatic coupling of the upper and lower gates may be weakened. However, a transistor including an ultra-thin channel layer according to a specific embodiment may achieve a large amplification factor while maintaining the electrostatic coupling. The electrostatic coupling of the upper and lower gates occurs when the upper channel interface is completely depleted. In an existing transistor, amplification may not occur because an electric field of a lower gate cannot control an upper channel.

The channel layer may include any one selected from the group consisting of an oxide semiconductor, an organic semiconductor, polycrystalline silicon, and monocrystalline silicon. When the channel layer includes any one selected from the group consisting of a semiconductor, an organic semiconductor, polycrystalline silicon, and monocrystalline silicon, electrostatic coupling of upper and lower gates may occur, a highly sensitive sensor may be manufactured, and a transparent and flexible sensor may be provided. A width or length of the channel layer is not limited, and electrostatic coupling may be utilized by using upper and lower gate electrodes in a dual-gate structure.

Also, in the sensor, an equivalent oxide thickness of the upper insulating layer may be thinner than a thickness of an equivalent oxide film of the lower insulating layer. For example, the thickness of the upper insulating layer may be about 25 nm or less, and the thickness of the lower insulating layer may be about 50 nm or more. When the thickness of the equivalent oxide film of the upper insulating layer is thinner than the thickness of the equivalent oxide film of the lower insulating layer, amplification of signal sensitivity may occur.

The upper insulating layer and the lower insulating layer may include a naturally or artificially formed oxide film. Examples of the oxide film include SiₓO_{y}, HₓfO_{y}, AlₓO_{y}, TaₓO_{y}, or TiₓO_{y} (wherein x or y is an integer of 1 to 5). The oxide film may have a single, double, or triple-layered structure. Thus, by increasing the physical thickness and decreasing the thickness of the equivalent oxide film of the upper insulating layer, the sensitivity of the sensor may be amplified, and deterioration thereof due to the leakage current may be prevented.

A dual-gate ion-sensitive field-effect transistor according to a specific embodiment may include both a field effect transistor including an upper insulating layer and a lower field effect transistor including a lower insulating layer in one device. Depending on respective modes of operation, each gate may independently be operated as an upper gate or a lower gate. When upper and lower gates of a device are used simultaneously, the electrostatic coupling may be observed due to the structural specificity of a dual-gate structure, and thus correlation between upper and lower field effect transistors may be established. In a dual operation mode, a lower gate may be used as a main gate. Thus, a transistor according to a specific embodiment may be operated in a dual-gate mode.

In another embodiment, the sensing unit may further include a probe coupled to the analyte-binding material via an analyte in a sample and having a negative charge or a positive charge. Signals of the analyte may be amplified by electrostatic coupling (capacitive coupling) of the probe to electrons in the channel layer of the transistor.

The probe may include metal nanoparticles. The metal nanoparticles may be, for example, gold nanoparticles, which may additionally supply charges. The probe may also include a quantum dot. When a quantum dot is used, the quantum dot may additionally supply charges as gold nanoparticles and also perform bioimaging at the same time. The probe may also include ferritin. The combined structure of ferritin and metal nanoparticles may provide larger signals by providing more charges than the single-metal nanoparticles.

In still another embodiment, the sensor may include a plurality of sensing units for detecting a plurality of analytes and a plurality of transistors.

The sensor may include the plurality of sensing units and the plurality of ISFETs, wherein the plurality of sensing pars may be electrically connected to the plurality of ISFETs, respectively. In the plurality of transistors, a plurality of sources may commonly be grounded, a plurality of upper gate electrodes may commonly be grounded, and a common voltage may be applied to a plurality of lower gate electrodes. For example, sources of a first transistor and a second transistor, and reference electrodes of a first sensing unit and a second sensing unit may commonly be grounded. For example, a common voltage may be applied to lower gate electrodes of the first transistor and the second transistor. In addition, a plurality of drains in the plurality of transistors may have a parallel structure. For example, drains of the first transistor and the second transistor may have a parallel structure. The plurality of sensing units may each independently include different immobilized analyte-binding materials. For example, an antibody against PSA may be immobilized on the first sensing unit, and an antibody against PSMA may be immobilized on the second sensing unit. The plurality of transistors may sense the same or different analyte signals from the plurality of sensing units, amplify the signals, and output the signals through a semiconductor parameter analyzer.

In still another embodiment, the signal processing unit may further include a calculation module for determining an amount of an analyte in a sample from a potential difference measured from the transistor, the calculation module being electrically connected to the transistor. The calculation module may be for the determination of an analyte. The term “determination of an analyte” as used herein may refer to a qualitative, semi-quantitative, or quantitative process for evaluating a sample. In a qualitative evaluation, the result indicates whether an analyte is detected in a sample. In a semi-quantitative evaluation, the result indicates whether an analyte is present above a predefined threshold value. In a quantitative evaluation, the result is a numerical indication of the amount of an analyte present therein. In order to convert measured values, a look-up table that converts a specific value of a current or a potential into a value of an analyte depending on a correction value for a specific device structure and an analyte may be used. The calculation module may determine an amount of an analyte by measuring a potential difference according to a known concentration of an analyte. For example, the calculation module may determine an amount of a colorectal cancer biomarker in a sample, as compared with that of a normal control group.

In still another embodiment, the sensor may include a communicator, which may allow the sensor to transmit/receive information to/from an external server or a terminal unit. The communicator may employ a wired or wireless communicator. Therefore, wired communication via a cable connection may be used, and wireless communication, including via a 4G, LTE, UWB, WiFi, WCDMA, USN, or IrDA module, as well as a Bluetooth module or a Zigbee module, may be used.

The terminal unit may include a communication device such as a computer, a notebook computer, a smartphone, a general mobile phone, a personal digital assistant (PDA), and a measuring instrument or a control device having a separate communication function. The terminal unit may include a central processing unit and may be based on an operating system (OS), capable of running software such as a computer program and an application program. Therefore, an application program, which is for reading, analyzing, and processing measurement data of an analyte in a sample provided by the sensor, may be mounted to the terminal unit, thus enabling the terminal unit to read, analyze, and process the measurement data of the analyte in the sample. The terminal unit may also display the measurement data of the analyte in the sample or the read, analyzed, and processed measurement data of the analyte in the sample. Also, the terminal unit may be connected to or linked with a control unit of the sensor, and thus the terminal unit may function to operate and control the sensor.

### Advantageous Effects of Disclosure

A sensor according to an aspect enables ultra-high precision/low-concentration detection of colorectal cancer biomarkers in a sample such as blood, stool, etc., thereby having an effect of enabling early diagnosis of colorectal cancer even with a very small amount of a sample.

### BRIEF DESCRIPTION OF DRAWINGS

FIG. 1 is a schematic diagram illustrating a sensor according to a specific embodiment;

FIG. 2 is a schematic diagram illustrating a sensing unit of the sensor according to a specific embodiment;

FIG. 3 is a schematic diagram illustrating signal amplification by a probe of the sensor according to a specific embodiment;

FIG. 4 shows results of evaluating stability of the sensor according to a specific embodiment;

FIG. 5 shows results of detecting CCSP2 in the actual sera of colorectal cancer patients using the sensor according to a specific embodiment; and

FIG. 6 is a graph showing results of actual serum samples of patients, a PDX model, and a control group.

### MODE OF DISCLOSURE

The terms used herein are those general terms currently widely used in the art in consideration of functions regarding the present embodiments, but the terms may vary according to the intention of those of ordinary skill in the art, precedents, or new technology in the art. Also, specified terms may be arbitrarily selected, and in this case, the detailed meaning thereof will be described in the detailed description. Thus, the terms used herein have to be defined not as simple names but based on the meaning of the terms and the overall description of the exemplary embodiments.

In descriptions of exemplary embodiments, it will be understood that when an element is referred to as being connected to another element, it may be directly connected to the other element or electrically connected to the other element with intervening elements therebetween. It will also be understood that when a component includes an element, unless there is another opposite description thereto, it should be understood that the component does not exclude another element and may further include another element. In addition, the terms “. . . unit” and “. . . module” as used herein refer to a unit that processes at least one function or operation and that may be embodied in a hardware manner, a software manner, or a combination of the hardware manner and the software manner.

The terms “configured” or “included” as used herein should not be construed to include all of various elements or steps described in the specification, and should be construed to not include some of the various elements or steps or to further include additional elements or steps.

The following description of the embodiments should not be construed as limiting the scope of the present disclosure, and modifications that those of skilled in the art may readily infer from the present disclosure should be construed as being within the scope of the present disclosure. Hereinafter, exemplary embodiments for descriptive sense only will be described in detail with reference to the accompanying drawings.

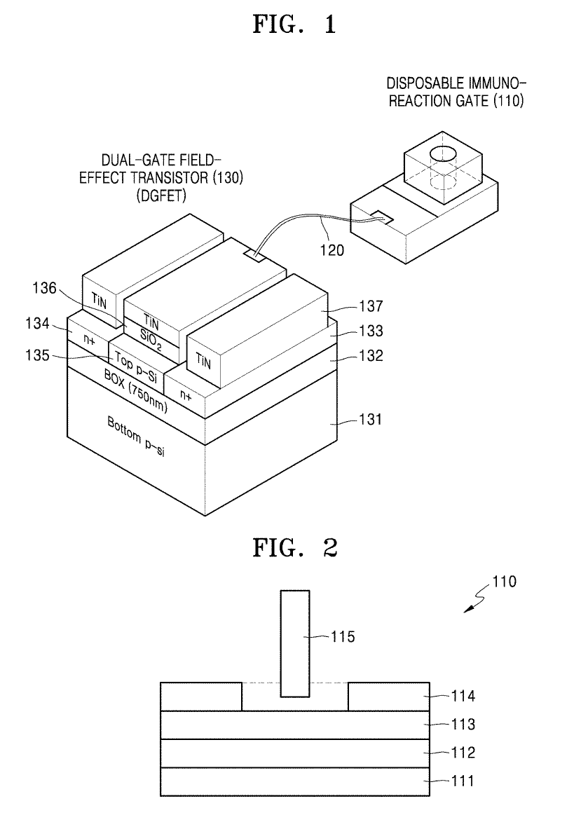

FIG. 1 is a schematic diagram illustrating a sensor according to a specific embodiment. Referring to FIG. 1, a sensor 100 according to a specific embodiment may include a sensing unit 110 for detecting an analyte in a sample and an ion-sensitive field-effect transistor 130 electrically connected to the sensing unit 110. In a specific embodiment, the sensor 100 may include the electrochemical sensing unit 110 for detecting an analyte in a sample and a signal processing unit 130 for amplifying signals generated from the sensing unit 110, wherein the signal processing unit 130 may be electrically connected to the sensing unit 110 and may include the ion-sensitive field-effect transistor 130, the sensing unit 110 may be separable from the signal processing unit 130, and the connection may be made between an electrode of the sensing unit 110 and an upper gate electrode of the transistor 130. In another specific embodiment, the sensor 100 may further include a connecting portion 120 for connecting the sensing unit 110 to the signal processing unit 130. The connecting portion 120 may be configured to be separable from the sensing unit 110 and, for example, the connecting portion 120 have a form of a plug. In still another specific embodiment, the sensor 100 may further include a display unit for displaying results. The display unit may further include a display displaying the results and a frame including at least one control interface (e.g., a power button, a scroll wheel, etc.). The frame may include a slot for into which a sensor may be inserted. The frame may include a circuit, and thus, when the frame is provided with a sample, the frame may apply an electrical potential or current to an electrode of the sensor. A suitable circuit that may be used in the meter may be, for example, an appropriate voltage meter capable of measuring the potential across the electrode. A switch may also be provided, which may be open when the electrical potential is measured or closed for measuring the current.

The ion-sensitive field effect transistor 130 may include a lower gate electrode 131; a lower insulating layer 132 formed on the lower gate electrode 131; a source 134 and a drain 133 formed on the lower insulating layer 132 and separated from each other; a channel layer 135 formed on the lower insulating layer 132 and arranged between the source 134 and the drain 133; an upper insulating layer 136 formed on the source 134, the drain 133, and the channel layer 135; and an upper gate electrode 137 formed on the upper insulating layer 136. Due to super capacitive coupling generated in the dual-gate ion-sensitive field-effect transistor (ISFET) 130 including the channel layer 135, a small surface potential voltage difference that occurs in the sensing unit may significantly amplify a threshold voltage variation of a lower field-effect transistor. In this regard, an amplification factor may be determined according to a thickness of the lower insulating layer 132, a thickness of the channel layer 135, a thickness of the upper insulating layer 136. As the thickness of the lower insulating layer 132 increases, and as the thickness of the upper insulating layer 136 and the thickness of the channel layer 135 decreases, the amplification factor may become larger. The channel layer 135 may be an ultra-thin film layer having a thickness, for example, of 10 nm or less, 9 nm or less, 8 nm or less, 7 nm or less, 6 nm or less, 5 nm or less, or 4 nm or less. The channel layer 135 may include any one selected from the group consisting of an oxide semiconductor, an organic semiconductor, polycrystalline silicon, and monocrystalline silicon. Also, in the sensor, a thickness of an equivalent oxide film of the upper insulating layer 136 may be thinner than a thickness of an equivalent oxide film of the lower insulating layer 132. For example, the thickness of the upper insulating layer 136 may be about 25 nm or less, and the thickness of the lower insulating layer 132 may be about 50 nm or more. When the thickness of the equivalent oxide film of the upper insulating layer 136 is thinner than the thickness of the equivalent oxide film of the lower insulating layer 132, amplification of signal sensitivity may occur. A dual-gate ion-sensitive field-effect transistor 130 according to a specific embodiment may include both a field-effect transistor including an upper insulating layer 136 and a lower field-effect transistor including a lower insulating layer 132 in one device. Depending on respective modes of operation, each gate may independently be operated as an upper gate or a lower gate. When upper and lower gates of a device are used simultaneously, electrostatic coupling may be observed due to the structural specificity of the dual-gate structure, and thus, the correlation between upper and lower field-effect transistors may be established. In a dual operation mode, a lower gate may be used as a main gate. Thus, a transistor according to a specific embodiment may be operated in a dual-gate mode.

In still another specific embodiment, the sensor may include a plurality of sensing units 110 and a plurality of transistors 130 for detecting a plurality of analytes. The sensor may include the plurality of sensing units 110 and the plurality of ion-sensitive field-effect transistors 130, wherein the plurality of sensing units 110 may respectively be electrically connected to the plurality of ion-sensitive field-effect transistors 130. In the plurality of transistors 130, a plurality of sources may commonly be grounded, a plurality of upper-gate electrodes may commonly be grounded, and a common voltage may be applied to a plurality of lower-gate electrodes. In addition, a plurality of drains in the plurality of transistors 130 may have a parallel structure. The plurality of sensing units 110 may each independently include different immobilized analyte-binding materials. The plurality of transistors 130 may sense the same or different analyte signals from the plurality of sensing units 110, amplify the signals, and output the signals through a semiconductor parameter analyzer.

In still another embodiment, the signal processing unit may further include a calculation module (not shown) for determining the amount of an analyte in a sample from a potential difference measured by the transistor 130, in which the signal processing unit may be electrically connected to the transistor 130. The calculation module may be for the determination of an analyte. The calculation module may determine the analyte by measuring a potential difference according to a known concentration of the analyte. For example, the calculation module may determine an amount of a colorectal cancer biomarker in a sample, as compared with that of a normal control group. In still another specific embodiment, the sensor 100 may include a communicator (not shown) which may allow to transmit/receive information to/from an external server or a terminal unit. The communicator may employ a wired or wireless communicator.

FIG. 2 is a diagram illustrating a sensing unit of a sensor according to a specific embodiment. Referring to FIG. 2, the sensing unit 110 may include a substrate 111; a working electrode 112 and a reference electrode 115 formed on the substrate; analyte-binding materials immobilized on the working electrode 112; and a test cell 114 for accommodating the electrodes 112 and 115, the analyte-binding materials, and an analyte. The sensing unit 110 may be configured to be disposable. For example, the substrate may be a material selected from the group consisting of silicon, glass, metal, plastic, and ceramic. The electrodes 112 and 115 may include, for example, silver, silver epoxy, palladium, copper, gold, platinum, silver/silver chloride, silver/silver ion, or mercury/mercuric oxide. The sensing unit 110 may also include an insulating electrode 113 formed on the substrate 111 or on the working electrode 112. The insulating electrode 113 may include a naturally or artificially formed oxide film. Examples of the oxide film include Si₃O_{y}, HₓfO_{y}, AlₓO_{y}, TaₓO_{y}, or TiₓO_{y} (wherein x or y is an integer of 1 to 5). Formation of the oxide film may be performed by a known method. For example, an oxide may be deposited on a substrate by liquid phase deposition, evaporation, or sputtering. The analyte-binding materials may include DNAs, RNAs, nucleotides, nucleosides, proteins, polypeptides, peptides, amino acids, carbohydrates, enzymes, antibodies, antigens, receptors, viruses, substrates, ligands, membranes, or a combination thereof. For example, the analyte-binding material may be an antibody capable of specifically binding to colorectal cancer secreted protein (CCSP) such as CCSP-2, or carcinoembryonic antigen (CEA), each of which is a diagnostic marker for colorectal cancer. Examples of the analyte may include antigens such as peptides (e.g., hormones) or haptens, proteins (e.g., enzymes), carbohydrates, proteins, drugs, agricultural chemicals, microorganisms, antibodies, and nucleic acids participating in sequence-specific hybridization with complementary sequences. More specific examples of the analyte may include CCSP such as CCSP-2, or CEA, each of which is a diagnostic marker for colorectal cancer. In the sensing unit 110, the sample may enter through the test cell 114 for accommodating the electrode, the analyte-binding material, and the analyte, and an analyte present in the sample may bind with the analyte-binding material to cause a chemical potential gradient in the test cell 114.

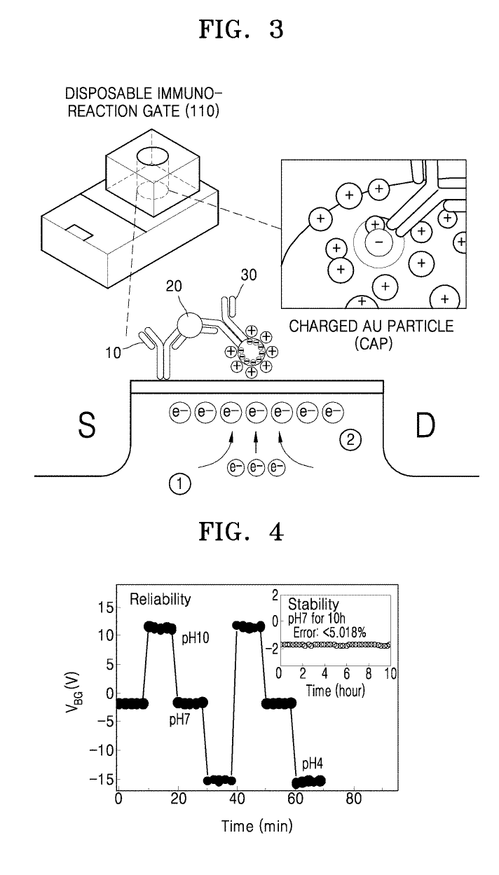

FIG. 3 is a schematic diagram illustrating a sensor using a probe according to a specific embodiment. Referring to FIG. 3, the sensing unit may further include a probe 30 that is coupled to analyte-binding materials 10 via an analyte 20 in a sample and has a negative charge or a positive charge. Charge collection (\textcircled{1}) may occur by the probe 30, and subsequently, signals of the analyte may be amplified by electrostatic coupling (capacitive coupling) (\textcircled{2}) of the probe 30 to electrons in the channel layer 135 of the transistor.

### EXAMPLE

### Manufacture of Sensor and Analysis of Characteristics

(1) Manufacture of Field-Effect Diagnostic Sensor for Colorectal Cancer

(1.1) Manufacture of Dual-Gate Ion-Sensitive Field-Effect Transistor

A silicon-on-insulator (SOI) substrate having resistivity of about 10 Ωcmto 20 Ωcm was prepared, a thickness of silicon as a lower-gate electrode was about 107 nm, and a thickness of a buried SiO₂ oxide film as a lower insulating layer was about 224 nm. After performing standard RCA cleaning, the upper silicon was etched with about 2.38% by weight of a tetramethylammonium hydroxide (TMAH) solution to form an ultra-thin film, and a channel region was formed by photolithography. In this case, a length, a width, and a thickness of the formed channel were respectively about 20 μm, about 20 μm, and about 4.3 nm. Subsequently, a titanium nitride (TiN) electrode was formed using a sputtering system. Then, an upper insulating layer was formed by oxidizing silicon dioxide having a thickness of about 23 nm on the source and the drain. To form an upper gate electrode, a TiN thin layer having a thickness of about 150 nm was deposited using a sputtering system. Next, to remove defects and improve an interfacial state therebetween, heat treatment was performed at a temperature of about 450° C. under a gas atmosphere including N₂ and H₂, thereby manufacturing a dual-gate ion-sensitive field-effect transistor.

(1.2) Manufacture of Electrochemical Sensing Unit

In order to prepare an electrochemical sensing unit, a glass of about 300 nm was used as a substrate. After standard RCA cleaning, a working electrode of ITO was deposited on the surface of the substrate at a thickness of about 100 nm using an E-beam evaporator to measure the electrical potential difference. Next, as an insulating electrode, a SnO₂ film which is an oxide film was deposited on the ITO layer to a thickness of about 45 nm using an RF sputtering method. At this time, RF power was about 50 W. Thereafter, a sputtering process was performed under an Ar gas atmosphere with a flow rate of about 20 sccm and a pressure of about 3 mtorr. Next, a test cell for accommodating a sample was prepared from polydimethylsiloxane (PDMS) and attached onto the insulating electrode to prepare a sensing unit. In addition, a silver/silver chloride electrode was used as a reference electrode.

(1.3) Manufacture of Sensor

A sensor was prepared by connecting the upper gate electrode of the transistor prepared in (1.1) to the working electrode of the sensing unit prepared in (1.2) in the form of a plug-in.

(2) Analysis of Characteristics of Sensor

(2.1) Evaluation of Sensor Stability

In order to evaluate stability of the sensor prepared in (1.3), pH 4, pH 7, and pH 10 solutions were alternately used. Further, a signal was measured at pH 7 for 10 hours to evaluate stability.

First, the pH 7 solution was reacted for 10 minutes, and then was removed. Subsequently, the pH 10 solution was injected and reacted for 10 minutes, and after the pH 10 solution was removed, the pH 7 sample was injected again and reacted for 10 minutes. Subsequently, the pH 4 solution was injected and reacted for 10 minutes. This process was repeated to analyze how the signals of the sensor varied. The results are shown in FIG. 4.

FIG. 4 is a graph showing the result of evaluating stability of the sensor according to a specific embodiment.

As shown in FIG. 4, it was found that even though different solutions were alternately injected into the sensor according to a specific embodiment, the reference voltage measured was consistent for each solution. Accordingly, the sensor according to a specific embodiment was found to stably measure electrical signals.

(2.2) Detection of Colorectal Cancer Marker CCSP2

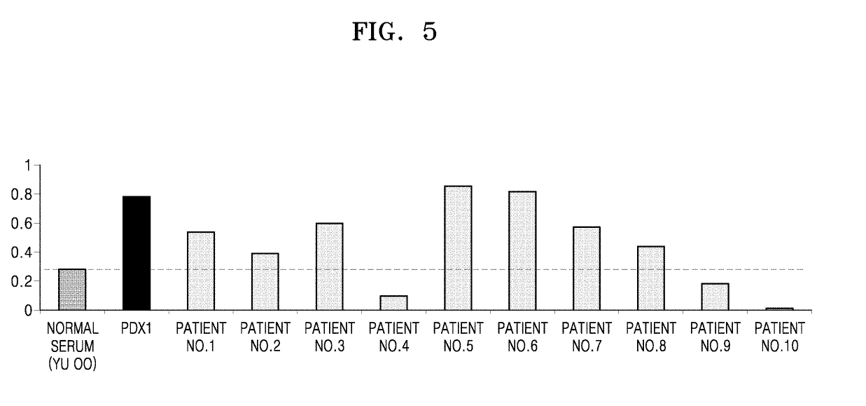

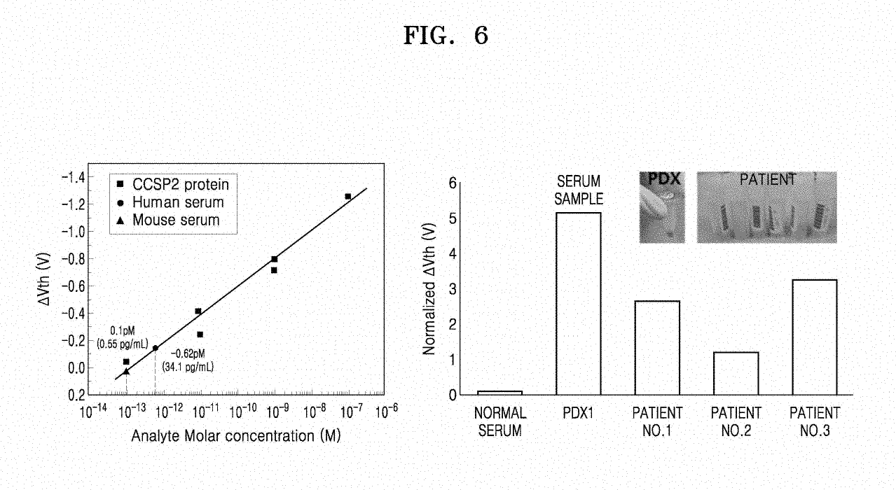

To detect a colorectal cancer marker CCSP2, thickness of Box (Buried oxide) and Top Si was adjusted to increase sensitivity for stable operation in the blood and stool. A disposable sensing membrane which was separated to protect the sensor was treated with colorectal cancer-specific antibodies to detect colorectal cancer markers, and the results are shown in FIGS. 5 and 6, and the following Table 1.

TABLE 1

| | Sample | | | | | |
|---|---|---|---|---|---|---|
| | Normal | PDX1 | No. 1 | No. 2 | No. 3 | No. 4 |
| ΔV | −0.1536 | 0.35356 | 0.10172 | −0.04471 | 0.16138 | −0.3362 |
| Concentration | 0.137 fg/ml | 1.36 fg/ml | 0.318 fg/ml | 0.137 fg/ml | 0.450 fg/ml | 0.0253 fg/ml |
| (fg/ml) | | | | | | |
| | Sample | | | | | |
| | No. 5 | No. 6 | No. 7 | No. 8 | No. 9 | No. 10 |
| ΔV | 0.4241 | 0.38574 | 0.14369 | 0.00409 | −0.25617 | −4.26E−01 |
| Concentration | 2.05 fg/ml | 1.64 fg/ml | 0.406 fg/ml | 0.181 fg/ml | 0.0402 fg/ml | 0.0151 fg/ml |
| (fg/ml) | | | | | | |

FIGS. 5 and 6 show the results of actual serum samples of patients and control groups.

As shown in FIGS. 5 and 6 and Table 1, the highest voltage variation was observed in the positive control, and higher positive voltage variations were observed in the sera of patients than that of a normal group, which were compared with standard data to quantitatively calculate CCSP-2 detection in the sera of patients.

These results suggest that CCSP-2 not detectable by an existing ELISA method may be detected by the sensor according to a specific embodiment, indicating superiority of the diagnostic sensor according to a specific embodiment.

1. A diagnostic sensor for colorectal cancer, the sensor comprising:
an electrochemical sensing unit for detecting an analyte in a sample, and a signal processing unit for amplifying signals generated from the sensing unit, the signal processing unit being electrically connected to the sensing unit and comprising an ion-sensitive field-effect transistor,
wherein the sensing unit is separable from the signal processing unit,
the ion-sensitive field-effect transistor comprises a lower gate electrode; a lower insulating layer formed on the lower gate electrode; a source and a drain, formed on the lower insulating layer and separated from each other; a channel layer formed on the lower insulating layer and arranged between the source and the drain; an upper insulating layer formed on the source, the drain, and the channel layer; and an upper gate electrode formed on the upper insulating layer, and
the connection is made between an electrode of the sensing unit and the upper gate electrode of the transistor.

2. The diagnostic sensor for colorectal cancer of claim 1, further comprising a connecting portion for connecting the sensing unit to the signal processing unit.

3. The diagnostic sensor for colorectal cancer of claim 1, further comprising a display unit for displaying results.

4. The diagnostic sensor for colorectal cancer of claim 1, wherein the sensing unit comprises a substrate; a working electrode and a reference electrode both formed on the substrate; an analyte-binding material immobilized on the working electrode; and a test cell for accommodating the electrodes, the analyte-binding material, and the analyte.

5. The diagnostic sensor for colorectal cancer of claim 4, wherein the sensing unit further comprises a probe coupled to the analyte-binding material via the analyte in the sample and having a negative charge or a positive charge, and signals of the analyte are amplified by electrostatic coupling (capacitive coupling) of the probe to electrons in the channel layer of the transistor.

6. The diagnostic sensor for colorectal cancer of claim 4, wherein the analyte-binding material is DNA, RNA, a nucleotide, a nucleoside, a protein, a polypeptide, a peptide, an amino acid, a carbohydrate, an enzyme, an antibody, an antigen, a receptor, a virus, a substrate, a ligand, a membrane, or a combination thereof.

7. The diagnostic sensor for colorectal cancer of claim 4, wherein the analyte-binding material is an antibody capable of specifically binding to colorectal cancer secreted protein (CCSP) or carcinoembryonic antigen (CEA), each of which is a diagnostic marker for colorectal cancer.

8. The diagnostic sensor for colorectal cancer of claim 5, wherein the probe comprises metal nanoparticles.

9. The diagnostic sensor for colorectal cancer of claim 1, wherein a thickness of an equivalent oxide film of the upper insulating layer is thinner than a thickness of an equivalent oxide film of the lower insulating layer.

10. The diagnostic sensor for colorectal cancer of claim 1, wherein a thickness of the channel layer is 10 nm or less.

11. The diagnostic sensor for colorectal cancer of claim 1, wherein the channel layer comprises any one selected from the group consisting of an oxide semiconductor, an organic semiconductor, polycrystalline silicon, and monocrystalline silicon.

12. The diagnostic sensor for colorectal cancer of claim 1, comprising a plurality of sensing units each identical to the sensing unit and a plurality of ion-sensitive field-effect transistors each identical to the ion-sensitive field-effect transistor, wherein the plurality of sensing units are electrically connected to the plurality of ion-sensitive field-effect transistors, respectively.

13. The diagnostic sensor for colorectal cancer of claim 12, wherein, in the plurality of transistors, a plurality of sources are commonly grounded, a plurality of upper gate electrodes are commonly grounded, and a common voltage is applied to a plurality of lower gate electrodes.

14. The diagnostic sensor for colorectal cancer of claim 12, wherein different analyte-binding materials are independently immobilized on each of the plurality of sensing units.

15. The diagnostic sensor for colorectal cancer of claim 1, wherein the signal processing unit further comprises a calculation module electrically connected to the transistor, the calculation module for determining an amount of the analyte in the sample from a potential difference measured by the transistor.
